(19) United States
(12) Patent Application Publication
(10) Pub. No.: US 2010/0015187 A1
(43) Pub. Date: Jan. 21, 2010

(54) MACRO-SIZED LIPID CAPSULE EMULSION COMPOSITION AND COSMETIC COMPOSITION CONTAINING THE SAME

(75) Inventors: Chan Koo Kang, Seoul (KR); Hak Hee Kang, Gyunggi-do (KR); Chang Hoon Park, Seoul (KR); Sang Hoon Han, Gyunggi-do (KR)

Correspondence Address: MERCHANT & GOULD PC, P.O. BOX 2903, MINNEAPOLIS, MN, 55402-0903, US

(73) Assignee: Amorepacific Corporation, Yongsan-gu (KR)

(21) Appl. No.: 12/300,251

(22) PCT Filed: Nov. 30, 2006
(86) PCT No.: PCT/KR2006/005111

§ 371 Date: May 21, 2009

(30) Foreign Application Priority Data
May 11, 2006 (KR) 10-2006-0042420

(51) Int. Cl. A61K 8/06; A61Q 5/00; A61Q 19/00

(52) U.S. Cl. 424/401

(57) ABSTRACT

An emulsion composition of the invention has macro-sized (0.01 to 5 mm) lipid capsule particles and it is for a skin cosmetic composition. An emulsion composition of the invention exhibits improved stability for its particle size by adjusting a size of the particle through a combination of liquid phase oil and integument lipid or lipoid material and encapsulating the liquid phase oil with the integument lipid. The emulsion composition of the invention has unique outward appearance and use feeling due to the macro-sized particles in it, also exhibits excellent skin safety and moisture persistence by containing no surfactant and maintains an activity of an oleophilic effective ingredient which is apt to be oxidized for a long time.

### TECHNICAL FIELD

The invention relates to an emulsion composition having macro-sized lipid capsule particles, and more particularly to an emulsion composition exhibiting improved emulsion stability for its particle size and having unique outward appearance, use feeling, excellent skin safety and moisture persistence by preparing a macro-sized particle having liquid phase oil trapped in a lipid capsule through an appropriate combination of the liquid phase oil and lipid ingredients for integument. In addition, the invention relates to an emulsion composition capable of preserving activity of its effective ingredient which is apt to be oxidized for a long time, by trapping the effective ingredient in macro-sized particles.

### BACKGROUND ART

In recent years, it has been developed basic and color cosmetics of various formulations. Among other things, it has been continuously carried out a research for giving a new merit using an emulsion formulation technology. The emulsion, which is a formulation system typically used in the cosmetics industry, is a system wherein an oil phase and a water phase are uniformly dispersed through emulsification. The factors influencing on physicochemical properties of the emulsion include types of surfactants, a ratio of inner and outer phases, emulsion types (oil/water (O/W), water/oil (W/O), water/oil/water (W/O/W) and the like), thickener, particle size, etc. Recently, it has been spotlighted an attempt to develop an emulsion formulation having new properties and efficacies by controlling a size of particle. In general, the O/W emulsion applied to the cosmetics consists of particles having a size of approximately 0.5˜10 μm. In recent years, as concerns of the nano technologies are increased, it has been actively conducted a research on the nano emulsion having an extremely small particle as a result of using a high-pressure emulsifier (R. H. Muller et al., European Journal of Pharmaceutics and Biopharmaceutics (2000) 50, 161). The nano emulsion is an emulsion having nanometer-sized particles. Due to a property that as the particle gets smaller, the long-term stability of it gets more improved. The nano emulsion can be appropriately applied to the cosmetics, so that the cosmetics, to which the nano emulsion is applied, can be easily seen around.

While the research for decreasing the particle size of the emulsion is actively carried out, a research on the emulsion having large particles is relatively not. Since it is possible to observe the particles of the emulsion having a large particle (0.01˜5 mm) with naked eyes, the emulsion has an external appearance distinct from the general emulsion. In addition, when it is applied to the skin, it takes much longer for the oil in the particle to reach the skin, so that the emulsion can give a unique use feeling different from the general emulsion. Additionally, when an effective ingredient which is apt to be oxidized is trapped in the macro particles, its contact to water is difficult. As a result, it is expected that the emulsion is useful to keep its effective ingredient active for a long time. Despite the several advantages, the reason that the relating research has not been actively conducted is as follows: as the particle becomes larger, the coagulation, flocculation or oswald ripening between the particles occurs easily, so that the long-term stability of the emulsion is deteriorated. Although some researchers developed an alternative system wherein micro capsules formed by using an interfacial polymerization, a spray drying, a solvent evaporation and the like are dispersed in the water <J. W. Kim et al., Langmuir (2001) 17, 5435>, it is not an emulsion formulation technology, strictly speaking. In addition, when it is applied as cosmetic materials, the insoluble capsule remains on the skin, thereby giving foreign body sensation. Further, during the manufacturing process, it is required an additional special apparatus in addition to the general emulsion manufacturing apparatus, so that it is not efficient economically.

### DISCLOSURE

### Technical Problem

In order to solve the above problems, the inventors tried to develop an emulsion composition having a lipid capsule particle of a macro size (0.01˜5 mm). The inventors mixed liquid phase oil and an lipid ingredient for integument in an appropriate ratio so as for the integument lipid to effectively encapsulate the oil ingredient so that the coagulation or flocculation between the particles did not occur even though the size of the particle was large. The inventors did not use insoluble ingredients, contrary to the conventional micro capsule, so that when applied to the skin, foreign body sensation was not caused. In addition, the inventors tried to make a non-surfactant system, contrary to the general emulsion system, to improve skin safety and moisture persistence of it, thereby increasing a value of it as the cosmetic material.

Accordingly, an object of the invention is to provide a cosmetic composition capable of giving a new value to users by using an emulsion composition containing macro-sized lipid capsule particles to give unique external appearance and use feeling and to improve the skin safety and moisture persistence of it.

Another object of the invention is to provide a cosmetic composition capable of stably maintaining an activity of its effective ingredient for a long time by trapping the effective ingredient which is apt to be oxidized in a macro particle having a relatively small contact surface to water phase.

### Technical Solution

In order to achieve the above objects, there is provided an emulsion composition comprising (i) liquid phase oil, and (ii) lipid capsule particles comprising an integument lipid ingredient trapping the liquid phase oil therein, wherein the integument lipid ingredient is one or more selected from a group consisting of lipid and lipoid materials which are solid state at room temperatures, and the lipid capsule particle has a diameter of 0.01˜5 mm.

According to an embodiment of the invention, the emulsion composition may further comprise a water phase thickener.

According to an embodiment of the invention, a content of the integument lipid ingredient may be 0.5˜10.0 wt % for a total composition weight. According to an embodiment of the invention, a content of the liquid phase oil may be 2.0˜30.0 wt % for a total composition weight.

According to an embodiment of the invention, a content of the water phase thickener may be 0.01˜5.0 wt % for a total composition weight.

According to an embodiment of the invention, the integument lipid ingredient may be selected from a group consisting of higher alcohol, fatty acid, lipid peptide and a mixture thereof.

According to an embodiment of the invention, the higher alcohol may be one or more selected from a group consisting of behenyl alcohol, cetyl alcohol, stearyl alcohol, glyceryl monostearate, cetostearyl alcohol and batyl alcohol.

According to an embodiment of the invention, the liquid phase oil may be one or more selected from a group consisting of hydrocarbon-type oil, silicone-based oil, triglyceride-type oil and ester-type oil.

According to an embodiment of the invention, the water phase thickener may be selected from a group consisting of carbomer, polyacrylamide, ammonium acryloyldimethyltaureate-VP copolymer, xanthan gum, cellulose ether, polyvinyl derivative, alginate, polyacrylate, sodium magnesium silicate and a mixture thereof.

According to an embodiment of the invention, the lipid capsule particle may trap one or more effective ingredients selected from a group consisting of a whitening increasing ingredient, a wrinkle improving ingredient, an anti-aging ingredient and an antioxidant ingredient therein.

A skin cosmetic composition of the invention contain the emulsion composition of the invention.

According to an embodiment of the invention, the composition may have a formulation of skin softener, astringent lotion, nutrient lotion, nutrient cream, massage cream, eye cream, eye essence, essence, cleansing cream, cleansing lotion, cleansing foam, cleansing water, pack, powder, body lotion, body cream, body essence, body cleansing agent, hairdye, shampoo, rinse, toothpaste, oral refresher, hair gel, hair tonic, lotion, ointment, gel, cream, patch or spray.

### ADVANTAGEOUS EFFECTS

According to the emulsion composition of the invention, it has macro-sized particles which can be seen with naked eyes. The liquid phase oil are encapsulated by the integument lipid, so that the coagulation or flocculation between the oil particles can be effectively prevented even despite the large particle size. In addition, it has unique external appearance and use feeling including both a fresh feeling and a moist feeling. Additionally, any surfactant is not used, emulsion composition is very safe for skin and can keep skin moist. Further, regarding the stability of the effective ingredient in it, the contact area of the emulsion particles to the water phase is remarkably decreased and the effluent to the water phase is effectively prevented, so that the remarkable effect of stabilizing the effective ingredients in it is exhibited, as compared to the conventional emulsion.

### DESCRIPTION OF DRAWINGS

FIG. 1 shows a comparison result between use feeling of emulsions depending on the particle sizes of them;

FIG. 2 shows a comparison result between moisture persistence of emulsions depending on whether they contain surfactant or not; and

FIG. 3 shows a comparison result between skin safety of emulsions depending on whether they contain surfactant or not.

### BEST MODEL

The emulsion composition of the invention comprises particles of a macro size (0.01˜5 mm). The reason of which is as follows: when the particle's diameter is smaller than 0.01 mm, it is difficult to achieve the effects of external appearance, use feeling and effective ingredient stabilization, which are given by the macro particles; when the diameter is larger than 5 mm, it is not smoothly and uniformly applied, which is due to the particles too large, so that it is not appropriate as the cosmetic materials.

In the emulsion composition of the invention, the lipid ingredient for integument is contained in an amount of 0.5˜10.0 wt %, preferably 1.0˜5.0 wt % for a total composition weight, in the O/W formulation. When the content is less than 0.5 wt %, the oil cannot be sufficiently encapsulated, so that the formulation stability is deteriorated. When the content exceeds 10.0 wt %, the hard cream formulation is formed due to the excessive lipid ingredient, so that the advantages of the appearance and use feeling, which are provided by the invention, cannot be obtained.

In the emulsion composition of the invention, the liquid phase oil ingredient is contained in an amount of 2.0˜30.0 wt %, preferably 5.0˜20.0 wt % for a total composition weight, in the O/W formulation. When the content is less than 2.0 wt %, the oil content is too little, so that the advantage of the emulsion formulation distinct from the general solubilized gel formulation cannot be obtained. When the content exceeds 30.0 wt %, the lipid ingredients cannot effectively encapsulate the oil, so that the long-term stability of the formulation is rapidly deteriorated.

In the emulsion composition of the invention, the water phase thickener is contained in an amount of 0.01˜5.0 wt % for a total composition weight. When the content is less than 0.01 wt %, the emulsion cannot have a viscosity capable of suppressing the cream phenomena. When the content exceeds 5.0 wt %, the viscosity of the emulsion is too high, so that the emulsion cannot be applied as the cosmetic material and the foreign body sensation is caused when applying it to the skin.

The emulsion composition of the invention comprises primarily one or more lipid ingredients for integument selected from a group consisting of lipid, which is solid state at the room temperatures, and lipoid materials, liquid phase oil and water phase thickener.

In the emulsion composition of the invention, the lipid ingredients for integument is preferably behenyl alcohol, cetyl alcohol, stearyl alcohol, cetostearyl alcohol, batyl alcohol, glyceryl monostearate and the like. Since the lipid ingredient for integument prevents the macro particles from being coagulated and combined due to the high melting temperature of it, it increases the stability of the emulsion and does not cause the foreign body sensation when the emulsion is applied to the skin.

In the emulsion composition of the invention, although most of the cosmetic oil ingredients can be applied as the liquid phase oil ingredient, one or more selected from hydrocarbon-type oil (for example, squalane), silicone-based oil (for example, dimethicone), triglyceride-type oil, ester-type oil and the like can be used as the liquid phase ingredient.

In addition, the water-soluble moisturizer, which is typically used as the cosmetic material, can be appropriately selected depending on the purposes and uses of each product.

The skin cosmetic composition using the emulsion composition of the invention can be used for all types of the cosmetics and the formulations thereof are not particularly limited. For example, the formulations include formulations of skin softener, nutrient lotion, cosmetic emulsion, essence, nutrient cream, pack, gel or a skin adhesive type cosmetic material, or transdermal administration type formulation.

### MODE FOR INVENTION

Hereinafter, the invention will be more specifically described with reference to embodiments and experimental examples. However, it should be noted that the invention is not limited thereto.

### Embodiments 1 and 2, Comparative Examples 1 and 2

### Preparations of Emulsion Compositions Comprising Macro-Sized Particles Having Different Integument Lipid Ingredients

Based on weight percents as shown in a table 1, the oil phase parts of items 1 to 5 were heated up to 70° C. and dissolved in a separate receptacle and then dispersed with a homogenizer, thereby preparing a oleophilic mixture. The water phase parts of items 6 to 9 were heated up to 70° C. and dissolved. Then the oleophilic mixture was added to the water phase parts, thereby causing emulsification. Finally, the emulsion composition was filled in the air-tight receptacle and cooled to the room temperature with a cooler.

TABLE 1

| | comparative | comparative | | |
|---|---|---|---|---|
| ingredients | example 1 | example 2 | embodiment 1 | embodiment 2 |
| 1. ceresin | 2.0 | | | |
| 2. microcrystalline wax | | 2.0 | | |
| 3. behenyl alcohol | | | 2.0 | |
| 4. glyceryl monostearate | | | | 2.0 |
| 5. squalane | 10.0 | 10.0 | 10.0 | 10.0 |
| 6. purified water | to 100 | to 100 | to 100 | to 100 |
| 7. glycerine | 10.0 | 10.0 | 10.0 | 10.0 |
| 8. carbomer | 0.1 | 0.1 | 0.1 | 0.1 |
| 9. neutralizer | proper | proper | proper | proper |
| | quantity | quantity | quantity | quantity |

### Experimental Example 1

### Stability of the Formulations, and Size and Shape of Particle in Them Depending on the Integument Lipid Ingredients

One day after the preparations, the respective samples obtained from the comparative examples 1 and 2 and the embodiments 1 and 2 were taken and reserved in a constant temperature bath under 45° C. for 30 days. After 30 days the stability of the formulations, and size and shape of particle in them are shown in a table 2.

TABLE 2

| | stability after 30 | particle size | |
|---|---|---|---|
| ingredients | days | (diameter, mm) | particle shape |
| comparative | no particle | — | — |
| example 1 | | | |
| comparative | partial coagulation | 2.0~4.0 | sharp |
| example 2 | between the | | acicular shape |
| | particles | | |
| embodiment 1 | stable | 0.5~1.0 | spheroidal shape |
| embodiment 2 | stable | 0.1~0.5 | spheroidal shape |

As shown in the table 2, the stability of the formulations is different depending on the integument lipid ingredients, wherein the lipid ingredients such as behenyl alcohol and glyceryl monostearate were more effective than the hydrocarbon-type wax ingredient when preparing the stable emulsion composition having the ‘macro-sized’ particles. It is expected that ceresin cannot stably form the particles due to a high crystallization ability of itself and the hydrocarbon-type wax induces the coagulation between the particles due to hyperhydration. In addition, it was confirmed that the particle size could be adjusted depending on the integument lipid ingredients.

### Embodiment 3, comparative example 3

### Preparations of Emulsion Composition Containing Effective Ingredients

The formulation compositions are shown in a table 3 and the preparation method is same as that of the comparative examples 1 and 2 and the embodiments 1 and 2. Retinol 10S (BASF CO., retinol (10%)/soybean oil (90%)), which is an additional material, was added to the oil phase mixture just before the emulsification. The PEG-100 stearate, which is a typical surfactant for cosmetics, was added to the oil phase mixture just before the emulsification.

TABLE 3

| ingredients | comparative example 3 | embodiment 3 |
|---|---|---|
| 1. behenyl alcohol | 1.5 | 2.0 |
| 2. PEG-100 stearate | 0.5 | 0.0 |
| 3. squalane | 9.0 | 9.0 |
| 4. retinol 10S | 1.0 | 1.0 |
| 5. purified water | to 100 | to 100 |
| 6. glycerine | 10.0 | 10.0 |
| 7. carbomer | 0.1 | 0.1 |
| 8. neutralizer | proper quantity | proper quantity |

### Experimental Example 2

### Experiment to Research on the Effective Ingredient Stabilization Depending on Particle Sizes

In recent years, as the customer's need for the functionality of the cosmetics increases, a research on containing the various functional ingredients in the formulation stably has been actively conducted. For example, retinol, which is the most known as the functional cosmetic ingredient, has a remarkable antioxidant effect. Therefore, when it is applied as the cosmetic ingredient, it exhibits a high wrinkle improvement effect. However, since retinol is apt to be oxidized due to light, oxygen, heat and the like and the stability for the hydrolysis is very poor, the activity thereof is rapidly decreased when it is applied to the formulation including the water.

Accordingly, the experiment was conducted for the purpose of examining how much retinol exhibited the effective ingredient stabilization effect when it was contained in the emulsion composition of the invention, as compared to the general emulsion.

When prepared as shown in the table 3, the formulation stability, the particle size and the retinol stability of the emulsion under 45° C. reservation condition are shown in a table 4.

TABLE 4

| | formulation | particle size | retinol stability |
|---|---|---|---|
| ingredients | stability after 30 days | (diameter, μm) | after 30 days |
| comparative | stable | 5 | 55% |
| example 3 | | | |
| embodiment 3 | stable | 500 | 94% |

The retinol stability in the emulsion composition or the invention after 30 days reservation at 45° C. was improved by 1.7 times, as compared to the general emulsion. The particle size of the comparative example 3 was 5 μm on average and the particle size of the embodiment 3 was 500 μm (0.5 mm) on average. Accordingly, when calculating the surface area of the particles, the surface area of the particles of the embodiment 3 was smaller by about 100 times than that of the comparative example 3. When the larger the particle size is, the smaller the surface area is relatively. Therefore, it was confirmed that when trapping the effective ingredient which is apt to be oxidized in the large particles of the emulsion, the effect of stabilizing the effective ingredient was achieved.

### Experimental Example 3

### Test for Comparing the Use Feeling of Emulsion Compositions Depending on the Particle Sizes

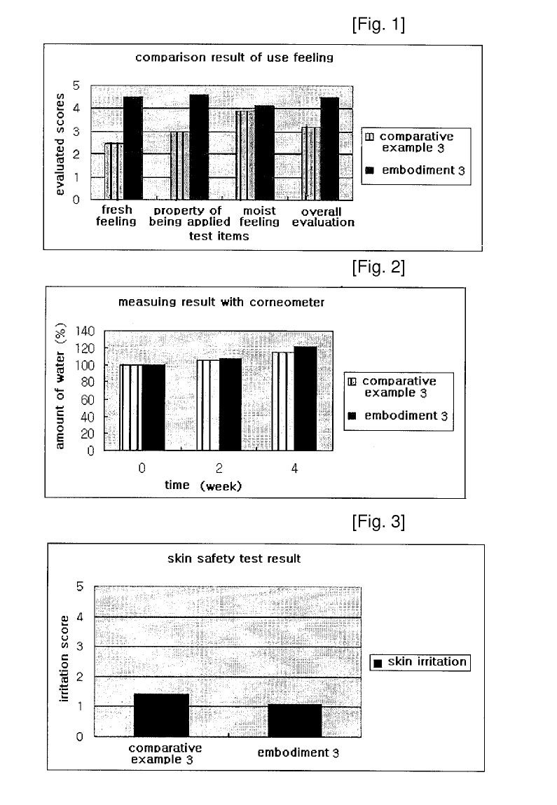

With the comparative example 3 and the embodiment 3, the test for use feeling was conducted for 30 female monitors whose ages were thirty to fifty. The test items were fresh feeling, property of being applied, moisture feeling and overall evaluation. Each of the items were given with scores of 0 to 5 and the average scores were shown in FIG. 1.

Referring to FIG. 1, the embodiment 3 achieved both the fresh feeling and the moist feeling, which are difficult to be achieved in the general emulsion. In general, the use feeling which the customer needs is the early light feeling and fresh feeling and the later strong moist feeling. However, in order to give the strong moist feeling, the oil ingredient should be typically contained in a large quantity. As a result, the early use feeling is heavy and the fresh feeling is not given.

The above result is obtained due to the following reasons: since the particles of the emulsion composition of the invention are larger by about 100 times than those of the general emulsion, it takes much longer for the oil particles to contact with the skin, so that the heavy feeling is decreased at early stage. Accordingly, even with the predetermined amount of the oil ingredient, the early fresh use feeling can be achieved and the strong moist feeling is also given after the particles are broken and the oils are applied to the skin.

### Experimental Example 4

### Test for Comparing the Moisture Persistence of Emulsion Compositions Depending on Whether they Contain the Surfactant or not

15 male and female adults with dry skin were divided into two groups. The adults in the respective groups applied the cosmetic compositions of the comparative example 3 and the embodiment 3 at their faces thereof two times every day, for 4 weeks. The water amounts of skin were measured with a corneometer under constant temperature and humidity condition (24° C., relative humidity 40%), before the application, 1 week, 2 weeks and 4 weeks after the application. The results are shown in FIG. 2. The corneometer is a device which measures and digitizes the ionization degrees of water using a sensor for the purpose of determining an amount of water (i.e., moisture ability) existing in the epidermis. The measuring method is as follows.

1) A probe of the comeometer is put on the skin to be measured.

2) When the probe is put on the skin, the capacitance of the skin is digitized through the sensor and displayed on a screen.

3) After the capacitance is measured, the sensor is wiped with paper such as kimwipe.

4) the measuring is repeated for the different parts.

As shown in FIG. 2, when the embodiment 3 was applied, the increase in the amount of water was much higher as time has lapsed, as compared to the case where the comparative example 3 was applied. This means that the moisture persistence of the embodiment 3 is superior. Since the embodiment 3 did not contain the surfactant, it has the superior waterproofing effect to the other emulsion formulations, thereby improved the moisture persistence.

### Experimental Example 5

### Test for Comparing the Skin Safety of Emulsion Compositions Depending on Whether they Contain the Surfactant or not

30 male and female adults with sensitive skin were asked to apply the cosmetic compositions of the comparative example 3 and the embodiment 3 at parts below the eyes so as to conduct the comparison test of the skin safety thereof. The irritation degrees were indicated with 0 point (no irritation) to 5 points (severe irritation) and the average thereof was shown in FIG. 3.

Referring to FIG. 3, the embodiment 3 induced much less irritation to the test subjects with sensitive skin than the comparative example 3 did. Since the embodiment 3 did not contain the surfactant, which has been known to cause the degeneration of protein and thus to induce the skin irritation, it is considered to exhibit the higher skin safety.

### INDUSTRIAL APPLICABILITY

According to the emulsion composition of the invention, it has macro-sized particles which can be seen with naked eyes. The liquid phase oils are encapsulated by the integument lipid, so that the coagulation or flocculation between the oil particles can be effectively prevented even despite the large particle size. In addition, it has unique external appearance and use feeling including both a fresh feeling and a strong moist feeling. Additionally, since any surfactant is not used, the emulsion composition is very safe for skin and can keep skin moist. Further, regarding the stability of the effective ingredient in it, the contact area of the emulsion particles to the water phase is remarkably decreased and the effluent to the water phase is effectively prevented, so that the remarkable effect of stabilizing the effective ingredients in it is exhibited, as compared to the conventional emulsion.

1. An emulsion composition comprising:
(i) liquid phase oil, and
(ii) lipid capsule particles comprising an integument lipid ingredient trapping the liquid phase oil therein,
wherein the integument lipid ingredient is one or more selected from a group consisting of lipid and lipoid materials which are solid state at room temperatures, and the lipid capsule particle has a diameter of 0.01˜5 mm.

2. The emulsion composition according to claim 1, further comprising a water phase thickener.

3. The emulsion composition according to claim 1, wherein a content of the integument lipid ingredient is 0.5˜10.0 wt % for a total composition weight.

4. The emulsion composition according to claim 1, wherein a content of the liquid phase oil is 2.0˜30.0 wt % for a total composition weight.

5. The emulsion composition according to claim 2, wherein a content of the water phase thickener is 0.01˜5.0 wt % for a total composition weight.

6. The emulsion composition according to claim 1, wherein the integument lipid ingredient is selected from a group consisting of higher alcohol, fatty acid, lipid peptide and a mixture thereof.

7. The emulsion composition according to claim 6, wherein the higher alcohol is one or more selected from a group consisting of behenyl alcohol, cetyl alcohol, stearyl alcohol, glyceryl monostearate, cetostearyl alcohol and batyl alcohol.

8. The emulsion composition according to claim 1, wherein the liquid phase oil is one or more selected from a group consisting of hydrocarbon-type oil, silicone-based oil, triglyceride-type oil and ester-type oil.

9. The emulsion composition according to claim 2, wherein the water phase thickener is selected from a group consisting of carbomer, polyacrylamide, ammonium acryloyldimethyltaureate-VP copolymer, xanthan gum, cellulose ether, polyvinyl derivative, alginate, polyacrylate, sodium magnesium silicate and a mixture thereof.

10. The emulsion composition according to claim 1, wherein the lipid capsule particle traps one or more effective ingredients selected from a group consisting of a whitening increasing ingredient, a wrinkle improving ingredient, an anti-aging ingredient and an antioxidant ingredient therein.

11. A skin cosmetic composition containing the emulsion composition according to claim 1.

12. The skin cosmetic composition according to claim 11, wherein the composition has a formulation selected from a group consisting of skin softener, astringent lotion, nutrient lotion, nutrient cream, massage cream, eye cream, eye essence, essence, cleansing cream, cleansing lotion, cleansing foam, cleansing water, pack, powder, body lotion, body cream, body essence, body cleansing agent, hairdye, shampoo, rinse, toothpaste, oral refresher, hair gel, hair tonic, lotion, ointment, gel, cream, patch and spray.
